(19) United States
(12) Patent Application Publication
(10) Pub. No.: US 2025/0009832 A1
(43) Pub. Date: Jan. 9, 2025

(54) COMPOSITION FOR INHIBITING PROLIFERATION OF TUMOR COMPRISING SANGUISORBA OFFICINALIS EXTRACT AS EFFECTIVE COMPONENT

(71) Applicant: KOREA INSTITUTE OF ORIENTAL MEDICINE, Daejeon (KR)

(72) Inventors: HWAN SUCK CHUNG, Daegu (KR); EUN-JI LEE, Gyeongsangbuk-do (KR); JANG GI CHOI, Daegu (KR); JI HYE KIM, Daegu (KR); YOUNG SOO KIM, Gyeonggi-do (KR); TAEIN KIM, Daegu (KR); WEI LI, Daegu (KR)

(21) Appl. No.: 18/289,284

(22) PCT Filed: May 3, 2022
(86) PCT No.: PCT/KR2022/006326

§ 371 Date: Sep. 16, 2024

(30) Foreign Application Priority Data
May 4, 2021 (KR) 10-2021-0057919

(51) Int. Cl. A61K 36/739; A61K 45/06; A61P 35/00

(52) U.S. Cl. CPC A61K 36/739; A61K 45/06; A61P 35/00

(57) ABSTRACT

The present invention relates to a composition for inhibiting proliferation of tumor comprising Sanguisorba officinalis extract as effective component. Specifically, as the Sanguisorba officinalis extract binds to PD-L1 present on the surface of cancer cells, the interaction between cancer cells and PD-1 on the surface of T cells is blocked to activate T cells, and thus an inhibitory effect on tumor proliferation is exhibited. Furthermore, when administered to an animal model induced with colorectal cancer, it is possible to obtain a decrease in tumor size and tumor weight and also the enhanced secretion of immune cells (CD8 T cells) and granzyme B in cancer tissues.

### TECHNICAL FIELD

The present invention relates to a composition for inhibiting proliferation of tumor comprising Sanguisorba officinalis extract as effective component.

### BACKGROUND ART

Conventional cancer treatments that are known to aim at conquering cancer typically involve surgery to remove tumor, along with the simultaneous use of radiation therapy and chemotherapy to reduce the tumor size before surgery or induce the death of remaining cancer cells and prevent recurrence after surgery. Radiation therapy and chemotherapy, which are referred to as the first-generation cancer therapies, include disrupting the process of uncontrolled infinite proliferation of cancer cells, leading to the induction of cancer cell death. However, radiation therapy poses a challenge as it induces not only DNA damage in cancer cells but also the death of normal cells through high-energy radiation. Moreover, chemotherapy, which involves administering toxic chemicals that hinder the cell division process, also has several problems as it does not selectively impede cancer cell division to hinder division of normal cells, leading to serious side effects such as decreased white blood cell count and hair loss. To solve these problems, there has been an expectation that the development of targeted anti-cancer agents, known as the second-generation anti-cancer agents which selectively attack only cancer cells while sparing normal cells, would reduce the side effects associated with the first-generation anti-cancer agents.

Meanwhile, targeted anti-cancer agents are characterized by selective inhibition of cancer cells by acting only on proteins that induce cancer. However, since the proteins inducing cancer and the proteins exhibiting therapeutic effects vary depending on the type of cancer, it is necessary to use anti-cancer agents that match the type of target protein. Additionally, as cancer cells possess mechanisms to acquire resistance to targeted anti-cancer agents, they can develop ‘immune evasion ability’ by having mutations to prevent themselves from being targeted by immune cells, leading to situations where the targeted anti-cancer agent may fail to recognize cancer cells. Therefore, to reduce the side effects and resistance issues associated with anti-cancer treatments, development of third-generation immunotherapeutic agents for cancer is underway, which can solve those problems and allow immune cells to remember and continuously attack cancer cells even after treatment is discontinued.

Immunotherapy for cancer is a treatment approach that stimulates the body's immune system to boost its self-immunity, enabling immune cells to target and attack cancer cells. Immunotherapeutic anti-cancer agent focuses on enhancing the function of immune cells, awakening their ‘potential to attack cancer cells.’ The immunotherapeutic anti-cancer agent can be categorized into immune checkpoint inhibitors, immune cell therapeutic agents, therapeutic antibodies, and anti-cancer vaccines. Immune checkpoint inhibitors are an anti-cancer agent for blocking the activity of immune checkpoint proteins that suppress T-cell function, thus activating T cells to attack cancer cells. Antibodies recognizing CTLA-4, PD-1, and PD-L1 are commonly used as an immune checkpoint inhibitor. Immune cell therapeutic agents are an anti-cancer agent for boosting cell-mediated immunity and they include natural killer (NK) cell therapeutic agents, T-cell therapeutic agents, and chimeric antigen receptor T-cell (CAR-T) therapeutic agents. As for the therapeutic antibodies, antibody-drug conjugates are used, where the antibody binds to cancer cells, and upon drug release, it attacks the cancer cells. Furthermore, anti-cancer vaccines involve administering to a cancer patient tumor-specific antigens present in cancer cells or protein/peptide molecules that can enhance overall immune responses within the patient, and they are an immunotherapy which aims to activate the immune system, making the internal immune functions more active to attack cancer cells.

Meanwhile, Sanguisorba officinalis refers to the roots of the plant Sanguisorba officinalis or Sanguisorba officinalis L. var. longifolis (Bert.) Yu et Li, belonging to the genus Sanguisorba in the family Rosaceae. The plant of the genus Sanguisorba has been used as a medicinal plant for over 2,000 years and is naturally found in temperate regions of the Northern Hemisphere, particularly in Asia, Western Europe, and North America.

Traditionally, Sanguisorba officinalis has been frequently used for its hemostatic properties, and pharmacological studies related to this have been reported. Among the components of Sanguisorba officinalis, terpene glycosides have demonstrated hemostatic effects, with Sanguisorba officinalis glycoside I reported as the most effective. Additionally, the total saponins in Sanguisorba officinalis are known to promote the proliferation and differentiation of megakaryocyte progenitors. Apart from its hemostatic action, Sanguisorba officinalis has also been reported to have hematopoietic effects. Recent studies have shown that the total saponins, as well as Sanguisorba officinalis glycoside I and II (ziyuglycoside I, II), from Sanguisorba officinalis can improve the survival of mouse bone marrow cells. Furthermore, according to several studies, Sanguisorba officinalis was shown to have anti-allergic effects. It inhibited systemic allergic reactions induced by compound 48/80 and suppressed plasma histamine concentration. It has been also reported that, in an OVA-induced asthma model, the ethanol extract of Sanguisorba officinalis reduces inflammatory cell infiltration and lowers Th2 cytokines and IgE concentrations.

As for the technology relating to Sanguisorba officinalis, in Korean Patent Application Publication No. 2006-0102621, a therapeutic agent for hypersensitive skin disorders containing Sanguisorba officinalis extract as active component is disclosed. Additionally, Chinese Patent Application Publication No. 108714149 discloses the use of active component of Sanguisorba officinalis in the preparation of an anti-tumor drug. However, there is no disclosure of the composition of the present invention for inhibiting proliferation of tumor comprising Sanguisorba officinalis extract as effective component.

### DETAILED DESCRIPTION OF THE INVENTION

### Technical Problems to be Solved

The present invention is devised under the aforementioned circumstances, and it provides a composition for inhibiting proliferation of tumor comprising Sanguisorba officinalis extract as effective component. It is found that the effective component of the present invention, i.e., Sanguisorba officinalis extract, binds to PD-L1 present on the surface of cancer cells, thus blocking the interaction between cancer cells and PD-1 on the surface of T cells to activate T cells and exhibit an inhibitory effect on tumor proliferation. It is also found that, by administering Sanguisorba officinalis extract to an animal model induced with colorectal cancer, it is possible to obtain a decrease in tumor size and tumor weight. Additionally, it is also found that the secretion of immune cells (CD8 T cells) and granzyme B can be enhanced by the extract in cancer tissues, and the present invention is completed accordingly.

### Technical Means for Solving the Problems

To achieve the purpose described in the above, the present invention provides a functional health food composition for inhibiting immune checkpoint comprising Sanguisorba officinalis extract as effective component.

The present invention further provides a pharmaceutical composition for preventing or treating a disorder relating to immune checkpoint inhibition comprising Sanguisorba officinalis extract as effective component.

The present invention further provides an anti-tumor pharmaceutical composition comprising an anti-cancer active agent and Sanguisorba officinalis extract as effective component.

The present invention still further provides an anti-cancer adjuvant comprising Sanguisorba officinalis extract as effective component.

### Advantageous Effect of the Invention

The present invention relates to a composition for inhibiting proliferation of tumor comprising Sanguisorba officinalis extract as effective component. Specifically, as the effective component of the present invention, Sanguisorba officinalis extract binds to PD-L1 present on the surface of cancer cells, thus blocking the interaction between cancer cells and PD-1 on the surface of T cells to activate T cells and exhibit an inhibitory effect on tumor proliferation. Furthermore, when administered to an animal model induced with colorectal cancer, it is possible to obtain a decrease in tumor size and tumor weight and also the enhanced secretion of immune cells (CD8 T cells) and granzyme B in cancer tissues.

### BRIEF DESCRIPTION OF THE DRAWINGS

FIG. 1 shows the result illustrating the concentration-dependent inhibition of PD-1/PD-L1 binding when PD-1-coated plates were treated with anti-PD-1 at concentrations ranging from 0.1 to 100 μg/mL and subsequently bound with PD-L1. The symbols *, **, and *** indicate a statistically significant decrease in PD-1/PD-L1 binding when treated with anti-PD-1 at concentrations of 0.1 to 10 μg/mL compared to the untreated group. Specifically, * denotes p<0.05, ** denotes p<0.01, and *** denotes p<0.001.

FIG. 2 shows the result illustrating the concentration-dependent inhibition of PD-1/PD-L1 binding when PD-L1-coated plates were treated with Sanguisorba officinalis extract at varying concentrations followed by the binding of PD-1. The symbols ** and *** indicate a statistically significant decrease in PD-1/PD-L1 binding when treated with Sanguisorba officinalis extract at concentrations of 5 to 50 μg/mL compared to the untreated group. Specifically, the symbols ** denotes p<0.01, and *** denotes p<0.001.

FIG. 3 shows the result of determining the blocking ability of Sanguisorba officinalis extract on PD-1:PD-L1 in which the test was carried out via luciferase assay using luciferase.

FIG. 4 shows the result illustrating (B) a change in body weight following (A) the administration of Sanguisorba officinalis extract of the present invention in an animal model of colorectal cancer. Vehicle represents the colorectal cancer-induced group without an administration of Sanguisorba officinalis extract, SER 100 mpk represents the group administered with 100 mg/kg of Sanguisorba officinalis extract, SRE 300 mpk represents the group administered with 300 mg/kg of Sanguisorba officinalis extract, αPD-1 represents the group administered with 5 mg/kg of αPD-1, and SRE 300 mpk+αPD-1 represents the group administered with both 300 mg/kg of Sanguisorba officinalis extract and 2.5 mg/kg of αPD-1.

FIG. 5 shows the result of determining (A) the tumor size and (B) the tumor weight, both 18 days after the administration of Sanguisorba officinalis extract of the present invention in an animal model of colorectal cancer. Vehicle represents the colorectal cancer-induced group without an administration of Sanguisorba officinalis extract, SER 100 mpk represents the group administered with 100 mg/kg of Sanguisorba officinalis extract, SRE 300 mpk represents the group administered with 300 mg/kg of Sanguisorba officinalis extract, αPD-1 represents the group administered with 5 mg/kg of αPD-1, and SRE 300 mpk+αPD-1 represents the group administered with both 300 mg/kg of Sanguisorba officinalis extract and 2.5 mg/kg of αPD-1. The symbols *, **, and *** indicate, in the groups administered with the Sanguisorba officinalis extract of the present invention, a statistically significant decrease in tumor size or tumor weight compared to the colorectal cancer-induced group (Vehicle), with * representing p<0.05, ** representing p<0.01, and *** representing p<0.001.

FIG. 6 shows the result of determining the tumor size 18 days after the administration of Sanguisorba officinalis extract of the present invention in an animal model of colorectal cancer. Specifically, the test groups are as follows: (A) Vehicle, (B) the group treated with 100 mg/kg Sanguisorba officinalis extract (SRE 100 mpk), (C) the group treated with 300 mg/kg Sanguisorba officinalis extract (SRE 300 mpk), (D) the group treated with 5 mg/kg αPD-1 (αPD-1), and (E) the group treated with 300 mg/kg Sanguisorba officinalis extract and 2.5 mg/kg αPD-1 (SRE 300 mpk+αPD-1).

FIG. 7 shows the result of determining the increased levels of immune cells (CD8 T cells) and granzyme B secretion in cancer tissues. The symbols *, **, and ** indicate a statistically significant increase in the secretion levels of immune cells (CD8 T cell) and granzyme B compared to the Vehicle, with * representing p<0.05, ** representing p<0.01, and *** representing p<0.001.

### BEST MODE(S) FOR CARRYING OUT THE INVENTION

The present invention relates to a functional health food composition for inhibiting immune checkpoint comprising Sanguisorba officinalis extract as effective component.

The Sanguisorba officinalis extract can be produced by a method including the following steps:
(1) carrying out extraction by adding an extraction solvent to Sanguisorba officinalis;
(2) filtering the extract of the step (1); and
(3) concentrating under reduced pressure the extract filtered in the step (2) followed by drying to give extract, but the method is not limited thereto.

The extraction solvent of the above step (1) is preferably selected from water, C1-C4 lower alcohol, and a mixture thereof. It is more preferably C1-C4 lower alcohol, and even more preferably 70% (v/v) ethanol, but not limited thereto.

With regard to the production method described in the present invention, any kind of common methods that are generally known as extraction method in the pertinent art, e.g., filtration, hot water extraction, impregnation extraction, extraction by reflux condensation, and ultrasonic extraction, can be used. It is preferable that the extraction is carried out by adding the extraction solvent in an amount of 1 to 20 times the weight of dried Sanguisorba officinalis. More preferably, the extraction solvent is added in an amount of 5 to 15 times the weight of dried Sanguisorba officinalis. The extraction temperature is preferably between 4° C. and 50° C., but it is not limited thereto. Furthermore, the extraction time is preferably between 0.5 hour and 10 hours and more preferably between 0.5 hour and 5 hours, but it is not limited thereto. It is preferable that the concentration under reduced pressure of the step (3) in the above method is preferably carried out by using a vacuum concentration apparatus or a vacuum rotary evaporator, but it is not limited thereto. Additionally, the drying can be preferably performed by drying under reduced pressure, drying under vacuum, drying under boiling, spray drying, or freeze-drying, but it is not limited thereto.

The Sanguisorba officinalis extract is characterized in that it targets PD-L1 or CD80 on cancer cells or targets PD-1 or CTLA-4 on T cells. The cancer cells may be preferably those derived from any one of liver cancer, lung cancer, breast cancer, melanoma, stomach cancer, colorectal cancer, rectal cancer, skin cancer, bladder cancer, prostate cancer, ovarian cancer, cervical cancer, thyroid cancer, kidney cancer, fibrosarcoma, and hematologic malignancy, but it is not limited thereto.

The functional health food composition of the present invention can be prepared by adding the Sanguisorba officinalis extract by itself or mixing it with other food product or other food component. The composition can be suitably used by following a common method. Type of the functional health food composition is not particularly limited. Examples of the food product to which the Sanguisorba officinalis extract of the present invention can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. The health beverage containing the composition of the present invention may include various flavorings or natural carbohydrates as additional ingredients, similar to regular beverages. The natural carbohydrates may include monosaccharides such as glucose and fructose, disaccharides such as maltose and sucrose, and polysaccharides such as dextrin and cyclodextrin, as well as sugar alcohols such as xylitol, sorbitol, and erythritol. As sweeteners, natural sweeteners such as taumatin and stevia extracts, or synthetic sweeteners such as saccharin and aspartame, can be used. The ratio of the natural carbohydrates is generally about 0.01 to 0.04 g, preferably about 0.02 to 0.03 g per 100 g of the composition of the present invention.

The functional health food composition of the present invention may further comprise various nutritional supplements, a vitamin, an electrolyte, a flavor, a coloring agent, pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated beverage. Other than those, fruit juice or fruit pulp for producing vegetable beverage may be additionally comprised. Ratio of the additives is, although not critically important, preferably selected from a range of between 0.01 part by weight and 2 parts by weight relative to 100 parts by weight of the composition of the present invention.

The present invention further relates to a pharmaceutical composition for preventing or treating a disorder relating to immune checkpoint inhibition comprising Sanguisorba officinalis extract as effective component.

The aforementioned disorder relating to immune checkpoint inhibition is preferably cancer, infection, sepsis, or immunosenescence, and more preferably cancer, but not limited thereto. The cancer is the same as those described above.

The present invention further relates to an anti-tumor pharmaceutical composition comprising an anti-cancer active agent and Sanguisorba officinalis extract as effective component.

The aforementioned anti-cancer active agent is preferably an anti-cancer agent or an immune checkpoint inhibitor, but not limited to them. Preferred examples of the anti-cancer agent include one or more selected from actinomycin D, bleomycin sulfate, daunomycin, daunorubicin, doxorubicin, epirubicin, idarubicin, mitomycin, mitomycin-C, mitramycin, irinotecan, camptothecin, novobiocin, epirubicin, dactinomycin, amsacrine, teniposide, etoposide, cisplatin, carboplatin, oxaliplatin, paclitaxel, docetaxel, gefitinib, erlotinib, and afatinib. Preferred examples of the immune checkpoint inhibitor include an anti-PD-L1 antibody, an anti-PD-1 antibody, an anti-CD80 antibody, and an anti-CTLA-4 antibody, but are not limited to them.

The pharmaceutical composition of the present invention can take various forms for oral or parenteral administration. When formulated, production is made by using commonly-used diluents or vehicles such as fillers, expanders, binders, wetting agents, disintegrants, or surfactants. For solid preparations intended for oral administration, they may include tablets, pills, powders, granules, capsules, etc., and these solid formulations are formulated by mixing one or more compounds with at least one vehicle such as starch, calcium carbonate, sucrose, lactose, gelatin, etc. Additionally, lubricants such as magnesium stearate, talc, etc. can be used in addition to simple vehicles. For liquid preparations intended for oral administration, they may include suspensions, solutions, emulsions, syrups, etc., and in addition to commonly used simple diluents like water and liquid paraffin, various vehicles such as wetting agents, sweeteners, flavors, preservatives, etc. may be included. For preparations intended for parenteral administration, they may include sterilized aqueous solutions, non-aqueous solvents, suspensions, emulsions, freeze-dried formulations, suppositories, etc. Examples of non-aqueous solvents and solvents for suspension include propylene glycol, polyethylene glycol, vegetable oils such as olive oil, injectable esters such as ethyl oleate, etc. Examples of suppository bases include Witepsol, Macrogol, Tween 61, cocoa butter, laurin, glycerol gelatin, etc.

The pharmaceutical composition of the present invention can be administered either orally or parenterally, and when administered parenterally, it can be applied by topical application on skin, or an intraperitoneal, rectal, intravenous, intramuscular, subcutaneous, intrathecal, or intracerebrovascular injection method can be preferably selected.

The pharmaceutical composition according to the present invention is administered in a pharmacologically effective amount. In the present invention, the expression “pharmacologically effective amount” refers to an amount sufficient to treat a disease with a reasonable benefit/risk ratio applicable to medical therapy. The effective dosage level can be determined based on various factors, including the type and severity of the patient's condition, the activity of the drug, sensitivity to the drug, administration time, route of administration, elimination rate, treatment duration, factors including other drugs that are simultaneously used, and other well-known factors in the medical field. The pharmaceutical composition of the present invention can be administered as an individual therapeutic agent or in combination with other therapeutic agents, sequentially or simultaneously with conventional therapies, and in a single dose or multiple doses. It is important to administer, with consideration of the aforementioned factors, the minimum effective amount that can achieve maximum efficacy without having any adverse effects, and this can be readily determined by a person who is skilled in the pertinent art.

The dosage of the composition of the present invention may vary in range depending on the patient's weight, age, gender, health condition, diet, administration time, method of administration, elimination rate, and severity of the disease. The daily dosage ranges, based on the amount of Sanguisorba officinalis extract, from 0.01 to 2,000 mg/kg. Preferably, it ranges from 30 to 500 mg/kg, and even more preferably from 50 to 300 mg/kg. The composition can be administered 1 to 6 times per day. The pharmaceutical composition of the present invention can be used either alone or in combination with surgery, radiation therapy, hormone therapy, chemotherapy, antibody therapy, and other treatment methods using biological response modifiers.

The present invention still further relates to an anti-cancer adjuvant comprising Sanguisorba officinalis extract as effective component.

The anti-cancer adjuvant may comprise one or more additional active ingredients that exhibit the same or similar functions as Sanguisorba officinalis extract. For clinical use, the anti-cancer adjuvant can be administered either orally or parenterally, and when administered parenterally, it can be applied by an intraperitoneal, rectal, subcutaneous, intravenous, intramuscular, intrathecal, intracerebrovascular, or thoracic injection method. The anti-cancer adjuvant may be used in form of common medicinal product.

The anti-cancer adjuvant can be used either alone or in combination with surgery, radiation therapy, hormone therapy, chemotherapy, and other treatment methods using biological response modifiers. The daily dosage of the anti-cancer adjuvant is about from 0.0001 to 100 mg/kg. Preferably, it ranges from 0.001 to 10 mg/kg, and it can be preferably administered once a day or divided into several doses, but may vary in range depending on the patient's weight, age, gender, health condition, diet, administration time, method of administration, elimination rate, and severity of the disease. The anti-cancer adjuvant of the present invention can be, when formulated for actual clinical administration, administered in various parenteral dosage forms. In the case of formulation, it is usually formulated using diluents or vehicles such as fillers, expanders, binders, wetting agents, disintegrants, surfactants, or the like. For preparations intended parenteral administration, sterilized aqueous solutions, non-aqueous solvents, suspensions, emulsions, freeze-dried formulations, suppositories, etc. are included. Examples of non-aqueous solvents and solvents for suspension include propylene glycol, polyethylene glycol, vegetable oils such as olive oil, injectable esters such as ethyl oleate, etc. Examples of suppository bases include Witepsol, Macrogol, Tween 61, cocoa butter, laurin, glycerol gelatin, etc.

Hereinbelow, the present invention is explained in greater detail in view of Examples. However, the following Examples are given only for more specific explanation of the present invention and it is evident to a person who has common knowledge in the pertinent art that the scope of the present invention is not limited by them.

### Example 1. Preparation of Sanguisorba officinalis Extract

Ten grams of dried Sanguisorba officinalis (Great Burnet) were added with 100 mL of 70% (v/v) ethanol as a solvent, and subjected to three reflux extractions over 2 hours. The resulting extract was concentrated under reduced pressure to obtain Sanguisorba officinalis extract.

### Example 2. Competitive ELISA Analysis on Interaction Between Sanguisorba officinalis Extract and PD-L1

Using competitive ELISA analysis, it was found that the Sanguisorba officinalis ethanol extract competitively binds to PD-1 in a concentration-dependent manner, blocking the interaction with PD-L1.

The competitive ELISA analysis was performed using a PD-1/PD-L1 inhibitor ELISA screening kit by following the manufacturer's instructions. The procedure involves coating a 96-well plate with recombinant human PD-1 (1 μg/mL dissolved in PBS, 100 μL/well) overnight. The plate was then washed with PBS containing 0.1% Tween (PBS-T) and blocked with 2% (w/v) BSA in PBS for 1 hour at room temperature. After another round of washing, biotinylated hPD-1 (0.5 μg/mL, 50 μL/well) was added to each well, and the plate was incubated for 2 hours at room temperature. Following three washes with PBS-T, streptavidin conjugated with HRP (0.2 μg/mL, 50 μL/well) was added to each well, and the plate was incubated for 1 hour. Following the incubation, after a final wash with 0.1% PBS-T three times, relative chemiluminescence was measured using a SpectraMax L luminometer.

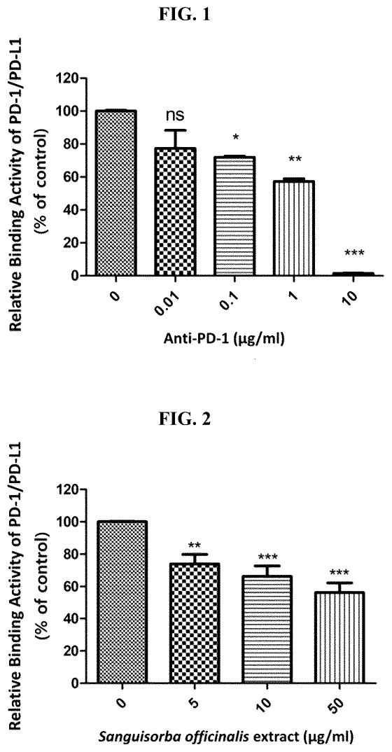

The results showed that the binding of PD-1/PD-L1 was inhibited in concentration-dependent manner (FIG. 1). Furthermore, Sanguisorba officinalis extract reduced, with statistical significance, the binding of PD-1/PD-L1 in a concentration-dependent manner (FIG. 2).

### Example 3. Determination of Sanguisorba officinalis Extract's Property of Blocking PD-1:PD-L1 Based on Reporter Assay Using Luciferase

To assess the inhibitory activity of Sanguisorba officinalis extract on PD-1/PD-L1 interaction, a cell-based PD-1/PD-L1 blockade reporter assay was conducted. The cell-based PD-1/PD-L1 blockade reporter assay takes an advantage of the principle that luciferase luminescence decreases when TCR binds to the TCR receptor due to PD-1/PD-L1 interaction. In other words, it is possible to determine with the assay whether the Sanguisorba officinalis extract of the present invention and PD-L1 blocking antibody block the PD-1/PD-L1 binding on the cell surface. Specifically, TCR/PD-1-overexpressing Jurkat T cells and TCR receptor/PD-L1-overexpressing CHO cells were used for the experiment. 1×104 PD-L1 aAPC/CHO-K1 cells were seeded in a 96-well plate and cultured in DMEM medium containing 10% FBS. On the day of the cell-based PD-1/PD-L1 blockade reporter assay, the medium was removed, and PD-1 blocking antibodies or Sanguisorba officinalis extract were added at various concentrations. Subsequently, 2×104 PD-1 overexpressing Jurkat T cells were added. After a certain period of time, Bio-Glo™ reagent (Promega) was mixed therein, and luminescence was measured.

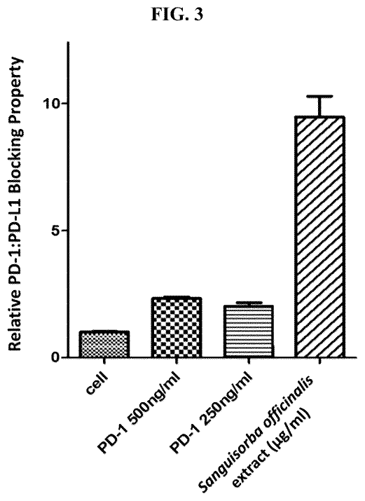

As a result, as it is illustrated in FIG. 3, when treated with Sanguisorba officinalis extract (100 μg/mL) and PD-1 blocking antibody (250 ng/ml of PD-1, 500 ng/ml of PD-1), a concentration-dependent inhibition of PD-1/PD-L1 binding was observed. Accordingly, an increase in the luminescence intensity of luciferase was shown.

### Example 4. Determination of Effect of Reducing Tumor Size as a Result of Administering Sanguisorba officinalis Extract Alone and in Combination with Anti-PD-1 (Sanguisorba officinalis Extract+Anti PD-1) Using Colorectal Cancer Animal Model

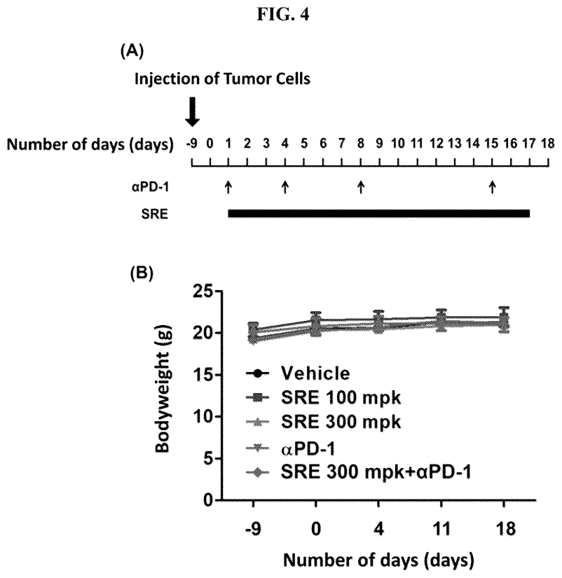

Using six C57BL mice in which mPD-1 was removed and substituted with hPD-1, MC-38 cells (mouse colorectal cancer cells) with mouse PD-L1 (mPD-L1) removed and replaced with human PD-1 (hPD-L1) were injected. As shown in the diagram of FIG. 4, αPD-1 (positive control) and Sanguisorba officinalis extract (100 and 300 mg/kg) were administered at intervals, and measurements of animal body weight and tumor size were carried out.

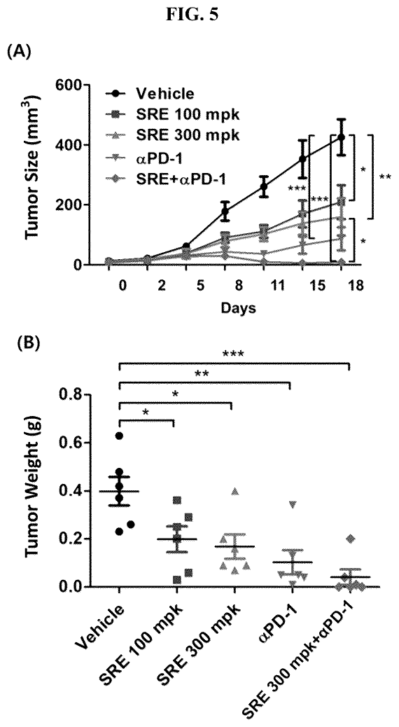

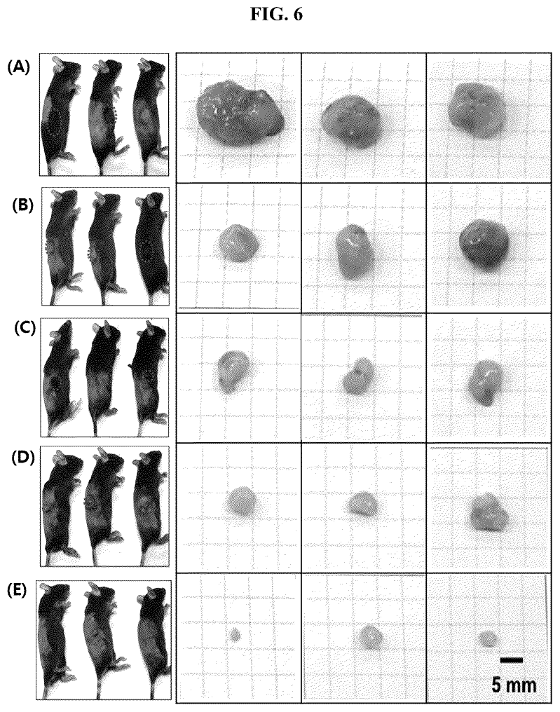

The results showed that there was no significant change in mouse body weight, but in comparison to the group with induced colorectal cancer (i.e., vehicle), the group treated with Sanguisorba officinalis extract of the present invention and the positive control group exhibited a significant decrease in tumor size and tumor weight (FIG. 5 and FIG. 6).

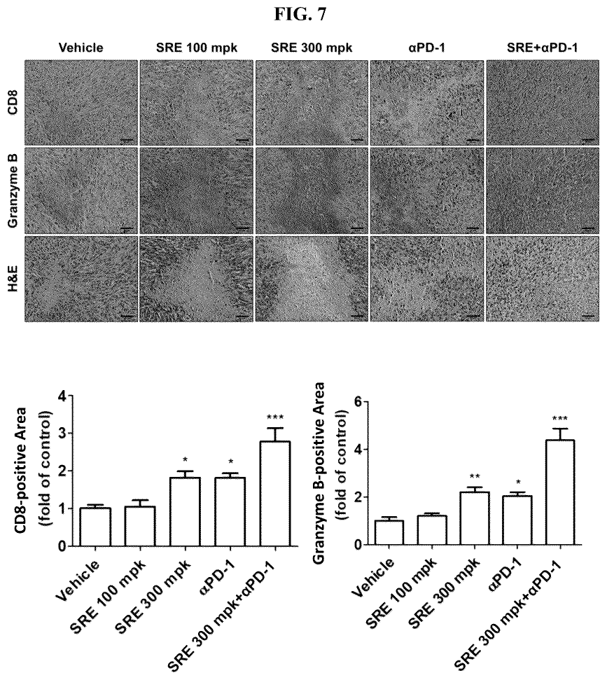

Furthermore, an increase in the secretion amount of immune cells (CD8 T cells) and granzyme B was observed in the tumor tissues of the animal model (FIG. 7).

### [Statistical Analysis]

In the present invention, the tests were repeated three times and the results are presented as mean±standard deviation. Statistical analysis was performed using Tukey's post-hoc test.

What is claimed is:

1. A functional health food composition for inhibiting immune checkpoint comprising Sanguisorba officinalis extract as effective component.

2. The functional health food composition according to claim 1, wherein an extraction solvent for obtaining the Sanguisorba officinalis extract is water, C1-C4 lower alcohol, or a mixture thereof.

3. The functional health food composition according to claim 1, wherein the Sanguisorba officinalis extract targets PD-L1 or CD80 on cancer cell or targets PD-1 or CTLA-4 on T cell.

4. The functional health food composition according to claim 3, wherein the cancer cell is that derived from any one of liver cancer, lung cancer, breast cancer, melanoma, stomach cancer, colorectal cancer, rectal cancer, skin cancer, bladder cancer, prostate cancer, ovarian cancer, cervical cancer, thyroid cancer, kidney cancer, fibrosarcoma, and hematologic malignancy.

5. A pharmaceutical composition for preventing or treating a disorder relating to immune checkpoint inhibition comprising Sanguisorba officinalis extract as effective component.

6. The pharmaceutical composition according to claim 5, wherein the disorder relating to immune checkpoint inhibition is cancer, infection, sepsis, or immunosenescence.

7. The pharmaceutical composition according to claim 6, wherein the cancer is selected from any one of liver cancer, lung cancer, breast cancer, melanoma, stomach cancer, colorectal cancer, rectal cancer, skin cancer, bladder cancer, prostate cancer, ovarian cancer, cervical cancer, thyroid cancer, kidney cancer, fibrosarcoma, and hematologic malignancy.

8. An anti-tumor pharmaceutical composition comprising an anti-cancer active agent and Sanguisorba officinalis extract as effective component.

9. The anti-tumor pharmaceutical composition according to claim 8, wherein the anti-cancer active agent is an anti-cancer agent or an immune checkpoint inhibitor.

10. The anti-tumor pharmaceutical composition according to claim 9, wherein the anti-cancer agent is one or more selected from actinomycin D, bleomycin sulfate, daunomycin, daunorubicin, doxorubicin, epirubicin, idarubicin, mitomycin, mitramycin, irinotecan, camptothecin, novobiocin, epirubicin, dactinomycin, amsacrine, teniposide, etoposide, cisplatin, carboplatin, oxaliplatin, paclitaxel, docetaxel, gefitinib, erlotinib, and afatinib.

11. The anti-tumor pharmaceutical composition according to claim 9, wherein the immune checkpoint inhibitor is an anti-PD-L1 antibody, an anti-PD-1 antibody, an anti-CD80 antibody, or an anti-CTLA-4 antibody.

12. An anti-cancer adjuvant comprising Sanguisorba officinalis extract as effective component.
